(19) United States
(12) Patent Application Publication
(10) Pub. No.: US 2009/0198132 A1
(43) Pub. Date: Aug. 6, 2009

(54) HAND-HELD ULTRASOUND IMAGING DEVICE HAVING RECONFIGURABLE USER INTERFACE

(76) Inventors: Laurent PELISSIER, Vancouver (CA); Kris DICKIE, Chilliwack (CA); Kwun-Keat CHAN, Vancouver (CA)

Correspondence Address: OYEN, WIGGS, GREEN & MUTALA LLP;480 - THE STATION, 601 WEST CORDOVA STREET, VANCOUVER, BC, V6B 1G1, CA

(21) Appl. No.: 12/188,191

(22) Filed: Aug. 7, 2008

Related U.S. Application Data
(60) Provisional application No. 60/955,328, filed on Aug. 10, 2007.

(51) Int. Cl. A61B 8/13

(52) U.S. Cl. 600/443

(57) ABSTRACT

A hand-holdable ultrasound machine has a number of user controls. The machine can be switched between at least a first mode wherein controls providing certain functions are in first locations and a second mode wherein the controls are in second locations. The machine may switch between modes in which the controls are positioned for convenient left- or right-handed operation and/or modes in which the controls are positioned for convenient one- or two-handed operation. The controls may be provided on a touch-sensitive display. A hand-holdable ultrasound machine displays images on a display. The images are rotatable. In some embodiments the machine senses a direction of motion and auto-rotates the images in response to the sensed direction.

### CROSS-REFERENCE TO RELATED APPLICATION

This application claims priority from U.S. patent application No. 60/955,328 filed on 10 Aug. 2007 and entitled HAND-HELD ULTRASOUND IMAGING DEVICE HAVING RECONFIGURABLE USER INTERFACE. This application claims the benefit under 35 USC §119 of U.S. patent application No. 60/955,328 filed on 10 Aug. 2007 and entitled HAND-HELD ULTRASOUND IMAGING DEVICE HAVING RECONFIGURABLE USER INTERFACE which is hereby incorporated by reference herein.

### TECHNICAL FIELD

This invention relates to ultrasound imaging devices. The invention relates particularly to ultrasound imaging devices having displays carried on hand-held units.

### BRIEF DESCRIPTION OF THE DRAWINGS

The accompanying drawings show non-limiting example embodiments of the invention.

FIG. 1 shows a hand-held ultrasound apparatus according to an embodiment of the invention.

FIGS. 2A and 2B show a display of the apparatus of FIG. 1 respectively in left-handed and right-handed operating modes.

FIGS. 3A and 3B show a hand-held ultrasound apparatus according to an embodiment of the invention having inputs located outside of a display.

FIGS. 4A and 4B show the apparatus of FIG. 1 with an ultrasound image rotated relative to the apparatus, but maintaining the same orientation relative to an operator.

FIGS. 5A and 5B show the apparatus of FIG. 1 respectively in two-handed and one handed operating modes.

FIG. 6 shows the apparatus of FIG. 1 with a virtual touch pad to allow the operator to select a specific position, or positions, on an ultrasound image.

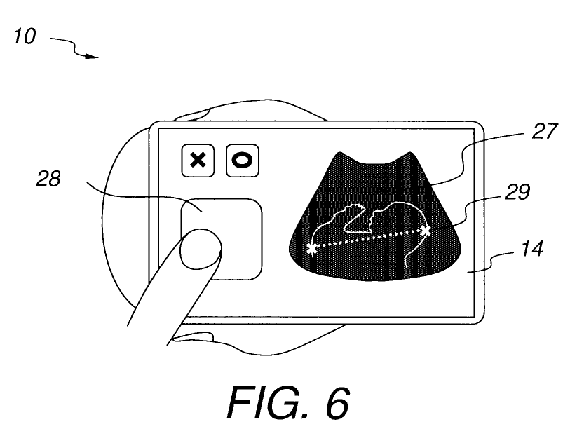

FIG. 7 is a block diagram showing functional elements of apparatus according to an example embodiment of the invention.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

The features as described herein may be combined in any suitable combinations with the features described in the following commonly-owned US provisional patent applications entitled:
HAND-HELD ULTRASOUND SYSTEM HAVING STERILE ENCLOSURE (application No. 60/955,327 filed 10 Aug. 2007);
HAND-HELD ULTRASOUND IMAGING DEVICE HAVING REMOVABLE TRANSDUCER ARRAYS (application No. 60/955,325 filed 10 Aug. 2007);
POWER MANAGEMENT IN PORTABLE ULTRASOUND DEVICES (application No. 60/955,329 filed 10 Aug. 2007);
WIRELESS NETWORK HAVING PORTABLE ULTRASOUND DEVICES (application No. 60/955,331 filed 10 Aug. 2007); and
HANDHELD ULTRASOUND IMAGING SYSTEMS (application No. 60/977,353 filed 3 Oct. 2007)
   all of which are hereby incorporated herein by reference. The features as described herein may also be combined in any suitable combinations with the features described in the commonly-owned US non-provisional patent applications which are filed on the same day as the instant application and entitled:
HAND-HELD ULTRASOUND SYSTEM HAVING STERILE ENCLOSURE (claiming priority from application No. 60/955,327 filed 10 Aug. 2007);
HAND-HELD ULTRASOUND IMAGING DEVICE HAVING REMOVABLE TRANSDUCER ARRAYS (claiming priority from application No. 60/955,325 filed 10 Aug. 2007);
POWER MANAGEMENT IN PORTABLE ULTRASOUND DEVICES (claiming priority from application No. 60/955,329 filed 10 Aug. 2007);
WIRELESS NETWORK HAVING PORTABLE ULTRASOUND DEVICES (claiming priority from application No. 60/955,331 filed 10 Aug. 2007); and
HANDHELD ULTRASOUND IMAGING SYSTEMS (claiming priority from application No. 60/977,353 filed 3 Oct. 2007)
   all of which are hereby incorporated herein by reference.

An ultrasound imaging device typically comprises a transducer having an array of transducer elements. The transducer elements are typically arranged in a pattern having a longitudinal axis and a transverse axis. For example, the transducer elements may be arranged in a line, in which case, the longitudinal axis extends along the line and the transverse axis is perpendicular to the longitudinal axis. Typically the transducer is longer than it is wide.

There are various protocols for performing ultrasonography. These protocols may require the transducer to be moved in different ways over the skin of a subject and held in different ways during the scanning. For example, for performing cardiac scanning it is typical to hold the transducer in the operator's left hand. Other types of scanning may be performed with the transducer held in the operator's right hand.

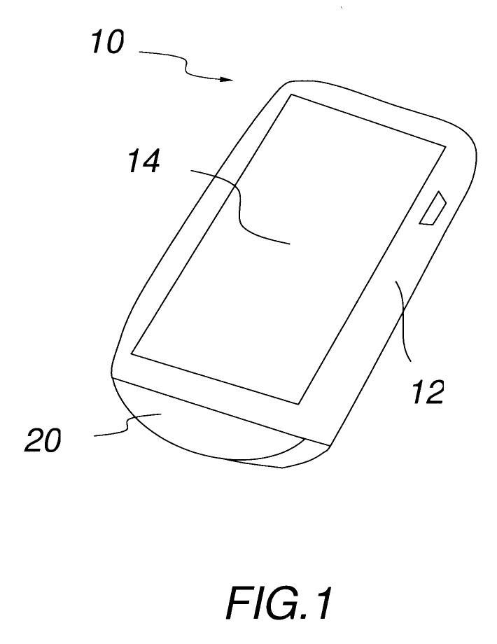

FIG. 1 shows a hand-held ultrasound unit according to an example embodiment of the invention. Hand-held ultrasound unit 10 may be used in either hand. Unit 10 comprises a transducer 20 and a housing 12 bearing a display 14. Display 14 is a touch-sensitive display. In some embodiments, substantially all of a front face of device 10 comprises a touch-sensitive display 14. Display 14 can display ultrasound images. In certain embodiments, display 14 also displays images representing controls with which an operator can interact to control the operation of apparatus 10.

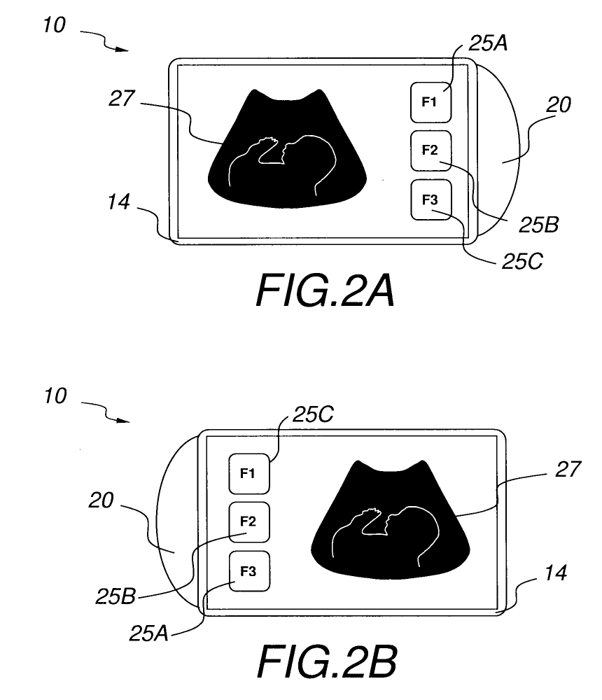

Apparatus 10 has a control, which may be a control displayed on screen 14, or which may comprise a switch, push button, or other input device located elsewhere on housing 12, which permits an operator to select between a right-handed operational mode and left-handed operational mode. The right-handed and left-handed operational modes differ in the location of controls displayed on display 14. It is typically convenient for the controls to be located close to transducer array 20. In the embodiment illustrated in FIGS. 2A and 2B, there are three controls 25A, 25B and 25C lined up along the edge of display 14 closest to transducer 20. Depending upon the nature of display 14 (i.e. the technology providing the touch-sensitive functionality of display 14) an operator can interact with the controls by touching, pressing, or holding a finger close to the image of the control on display 14. Display 14 senses the interaction with the operator's finger and actuates the control. In one embodiment, the order of controls 25A, 25B and 25C is reversed upon switching from the left-handed mode to the right-handed mode, as illustrated in FIGS. 2A and 2B. This permits the operator to comfortably use apparatus 10 in either hand.

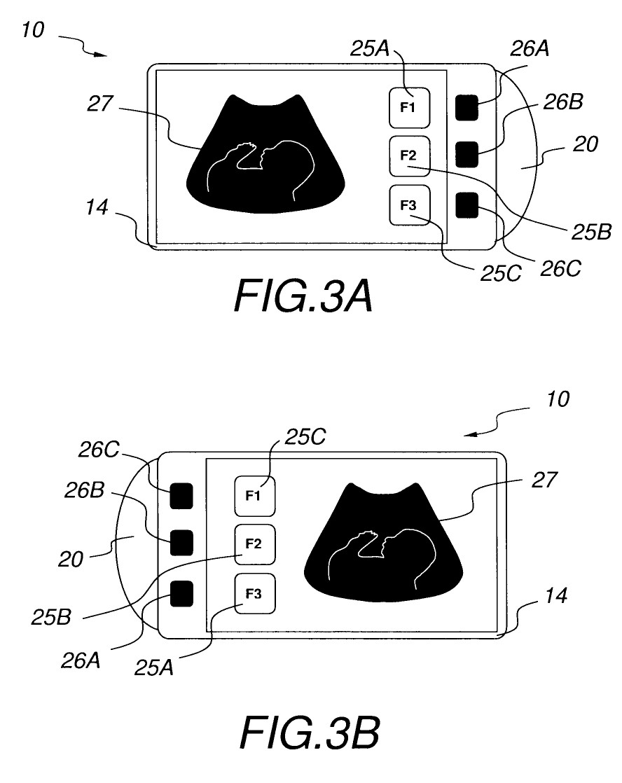

In an alternative embodiment of which FIGS. 3A and 3B show an example, the functions of controls 25A, 25B and 25C may be assigned to pushbuttons, switches, contact sensors, or the like 26A, 26B and 26C that are adjacent to but outside of display 14. In such embodiments, switching between left- and right-handed modes may switch the functions invoked by actuating inputs 26A, 26B, and 26C and may also change corresponding labels 27A, 27B, and 27C that are displayed on display 14 adjacent to inputs 26A, 26B and 26C.

Of course, the invention is not limited to the case where there are three controls affected by the operating mode. The invention may be applied to cases where there are any number of controls. Switching between left- and right-handed modes may move the locations of one or more controls.

In some scanning protocols it is necessary to move transducer 20 over the surface of a subject such that a longitudinal axis of transducer 20 extends more-or-less transversely to the direction of motion. In other scanning protocols it is necessary to move transducer array 20 such that the direction of motion of transducer array 20 across the subject is essentially parallel to the longitudinal axis of transducer array 20. In some embodiments, display 14 displays a ultrasound image 27. It is desirable that displayed ultrasound image 27 always have the same orientation relative to the operator such that the operator can readily comprehend ultrasound image 27.

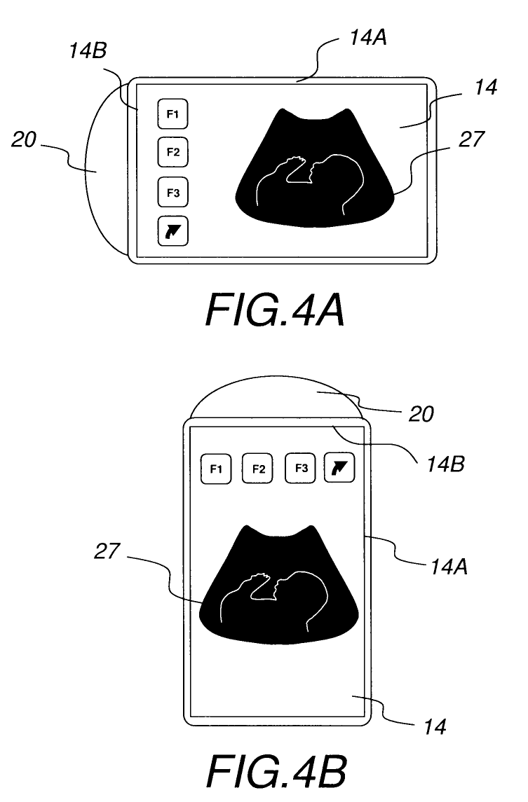

Apparatus according to the invention may permit the orientation of ultrasound image 27 to be changed to suit the scanning protocol being used. For example, ultrasound image 27 may have a first orientation relative to display 14 in which the top edge of ultrasound image 27 is adjacent to the top edge 14A of display 14, which extends away from transducer array 20 (as shown in FIG. 4A) and a second orientation such that the top edge of the ultrasound image is rotated by 90° relative to the first orientation and is aligned generally with a side edge 14B of display 14 closest to transducer array 20 (as shown in FIG. 4B). Switching between these orientations may be performed in various ways. For example, an operator may be able to select between these orientations in order to maintain the top edge of the display in a consistent position relative to the operator, as illustrated in FIGS. 4A and 4B.

In some embodiments, ultrasound apparatus 10 detects its direction of motion relative to the subject by analyzing the ultrasound data reflected back to transducer array from the subject. By doing this, ultrasound apparatus 10 can determine whether the direction of motion is parallel to the longitudinal axis of array 20 or more or less perpendicular to the longitudinal axis of array 20. This may be achieved, for example, by performing a two-dimensional correlation between successive frames of the ultrasound image acquired by way of transducer array 20. Such a correlation may be performed on multiple frames to verify the direction of motion. The existence of a correlation (e.g. where a computed correlation exceeds a threshold value) indicates scanning in a transverse direction relative to transducer array 20. the absence of a correlation indicates scanning parallel to the longitudinal axis of transducer array 20. In such embodiments, apparatus 10 may be configured to automatically orient the ultrasound image such that its top edge is away from the operator.

In other embodiments, a control, which may be provided on display 14 or may be a separate input may be provided to allow an operator to selectively rotate the ultrasound image so that it is properly oriented for the type of procedure being performed. In still further embodiments, an optical sensor is provided to detect the direction of motion of transducer array 20 over the subject. The optical sensor may, for example, comprise an optical sensor of the type used in an optical computer mouse. Apparatus 10 may automatically set the orientation of image 27 based at least in part upon the direction of motion sensed by the optical sensor.

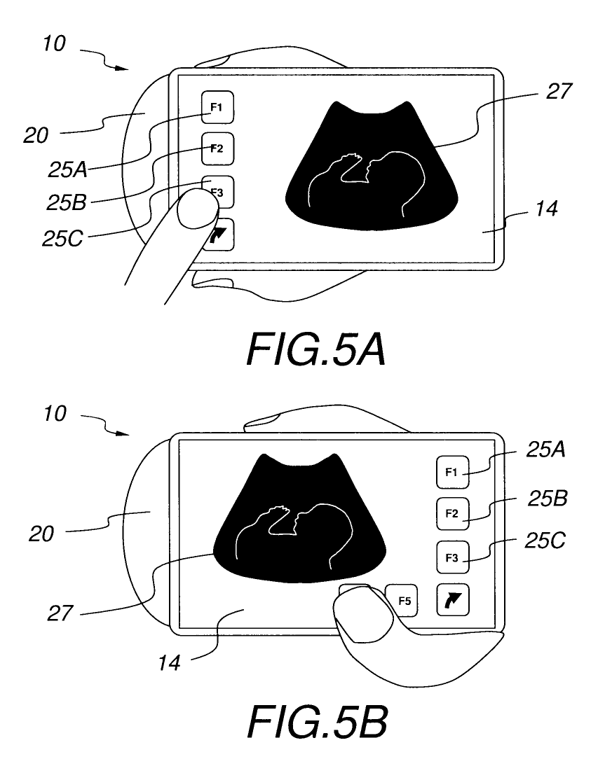

In further embodiments of the invention illustrated in FIGS. 5A and 5B, an operator can selectively configure touch screen 14 for either one-handed operation or two-handed operation. In the one-handed operation mode, user controls are located where they can be reached with the thumb or fingers of the same hand that the operator is using to grasp apparatus 10. In the two-handed operating mode, the user controls are located such that they can be conveniently operated by the operator's other hand (i.e. the hand that is not currently not holding housing 12).

Optionally, one or more physical buttons or other controls may be provided on housing 12 that an operator can use to make various inputs to apparatus 10. In some embodiments, the function performed by activating an input is software-configurable. In some such embodiments, the current function of the input may be displayed in a label displayed on display 14 adjacent to the location of the input in question.

In some cases it is desirable to permit an operator to indicate a specific position, or positions, on an ultrasound image being displayed on display 14. This functionality may be applied for various purposes. For example:
A specific position may be selected for performing Doppler ultrasound to measure blood flow, or a heart rate, or the like.
Distances between different points on an ultrasound image may be measured by specifying the two points and automatically computing the distance between the two points.
   In some embodiments of the invention, where display 14 comprises a touch screen, indicating a position on ultrasound image 27 is performed by way of a virtual touch pad 28 defined on a portion of screen 14 that is outside of ultrasound image 27. Virtual touch pad 28 may comprise an image defining an area on display 14. The operator can move a cursor 29 over image 27 by sliding a finger back and forth, or up and down on virtual touch pad 28. Device 10 detects motions of the operator's finger and adjusts the position of the current cursor 29 on display 14 in response thereto. This permits the operator to accurately specify a location on ultrasound image 27 without obscuring ultrasound image 27 with a finger.

In some embodiments of the invention, apparatus 10 may acquire and store a sequence of ultrasound images that can be played back by apparatus 10 as a moving picture or cineloop. In such embodiments, apparatus 10 may provide a user control on display 14 that permits control over playing of the cineloop or locating specific portions of the cineloop. For example, with the image frozen, the operator may navigate through a cineloop by sliding a finger over display 14. Sliding the finger in one direction, for example from left-to-right, may advance the cineloop while sliding the finger in an opposing direction, for example from right-to-left, may go back to earlier frames in the cineloop, or vice versa.

Instead of detecting sliding in one direction or another, device 10 may be configured to detect whether a finger is being moved in a clockwise or counterclockwise pattern over display 10 and to play a cineloop forward or backward depending upon the sense of rotation of the finger around display 10 or an area thereon. The rate that the cineloop is played may be set based upon a speed of motion of the operator's finger detected on display 14.

Other commands may be given by tapping or double-tapping on display 14. For example, apparatus 10 may be configured so that when an image is frozen, a double-tap on the image will cause apparatus 10 to store the image. In the alternative, apparatus 10 could be programmed so that a double tap freezes/unfreezes a cineloop or so that a double tap causes the current image to be printed or the like.

In some embodiments, apparatus 10 may be configured to recognize patterns or gestures drawn on display 10 and to associate specific patterns or gestures with commands. The patterns or gestures could be in the shapes of letters of the alphabet although this is not mandatory. For example:
An operator could draw a letter C on display 14 to invoke a COLOR command, an S to invoke a SAVE command, a P to invoke a PRINT command, etc.
   Such writing short cuts to invoke functions may be drawn anywhere on display 14.

The various features described above may be used together in any suitable combinations or sub-combinations. For example, an apparatus 10 could combine all of the following features:
selectable left-handed and right-handed operating modes;
selectable one-handed and two-handed operating modes;
manual and/or automatic reorientation of ultrasound images;
a touch pad or virtual touch pad that allows an operator to specify a point or points on an image;
a cineloop playback facility controlled by finger touch;
input of commands by way of simple gestures or patterns.
   Instead of providing all of the above features, apparatus 10 may provide any desired subset of these features.

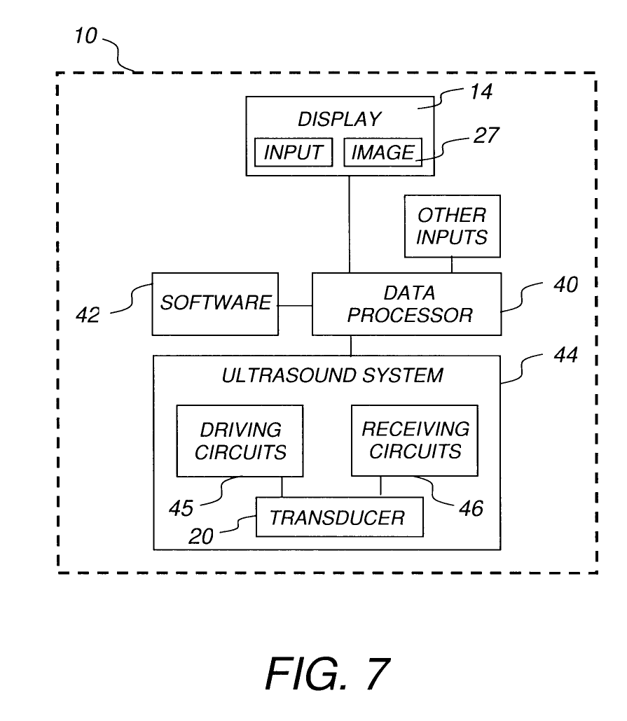

There are many possible ways to provide apparatus 10 that has features as described above. FIG. 7 shows a possible construction. In the embodiment of FIG. 7, apparatus 10 comprises a data processor 40 that may comprise, for example, a microprocessor, microcontroller, digital signal processor or the like. Data processor 40 executes software 42. Data processor 40 is connected to generate an image 27 on display 14 and is also connected to receive inputs from display 14 as well as from any other inputs provided by apparatus 10. Data processor 40 is also connected to control an ultrasound system 44 that comprises transducer 20, driving circuits 45 for driving elements of transducer 20 to emit ultrasound, and receiving circuits 46 that receive signals representing reflected ultrasound received at elements of transducer 20 and process those signals. Driving and receiving circuits 45 and 46 may comprise discrete components, application specific integrated circuits (ASICs), field programmable gate arrays (FPGAs), suitable data processors or suitable combinations thereof. A wide range of circuits suitable for use in ultrasonography are known to those skilled in the field of the invention. Software executing on data processor 40 may be included in ultrasound system 44.

Under control of software 42, data processor 40 receives data from ultrasound system 44 and selectively displays the data as an image 27 or otherwise on display 14. Data processor 40 also displays any controls on display 14. The selection of controls (and/or labels for controls) to be displayed on display 14 and the locations in which those controls are displayed will depend upon the current operating mode of apparatus 10.

Certain implementations of the invention comprise computer processors which execute software instructions which cause the processors to perform a method of the invention. For example, one or more processors in a hand-held ultrasound apparatus may implement methods as described herein executing software instructions in a program memory accessible to the processors. The invention may also be provided in the form of a program product. The program product may comprise any medium which carries a set of computer-readable instructions which, when executed by a data processor, cause the data processor to execute a method of the invention. Program products according to the invention may be in any of a wide variety of forms. The program product may comprise, for example, media such as magnetic data storage media including floppy diskettes, hard disk drives, optical data storage media including CD ROMs, DVDs, electronic data storage media including ROMs, PROMs, flash RAM, or the like. The computer-readable instructions on the program product may optionally be compressed or encrypted.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

While a number of exemplary aspects and embodiments have been discussed above, those of skill in the art will recognize certain modifications, permutations, additions and sub-combinations thereof.

What is claimed is:

1. A hand-holdable ultrasonic imaging device comprising:
a hand-holdable housing supporting a transducer array;
a display supported on the housing; and,
control circuits comprising a programmable data processor in the housing, the control circuits configured to control operation of the ultrasonic imaging device based at least in part on a user input corresponding to a first function;
wherein:
the imaging device has a first mode wherein the first function is assigned to a first user control supported on the housing at a first location; and
the imaging device has a second mode wherein the first function is assigned to a second user control supported on the housing at a second location.

2. A hand-holdable ultrasonic imaging device according to claim 1 wherein:
the first function is one of a plurality of functions,
the control circuits are configured to control operation of the ultrasonic imaging device according to user inputs corresponding to the plurality of functions,
in the first mode the plurality of functions are assigned to user controls at corresponding first locations on the housing,
in the second mode the plurality of functions are assigned to user controls at corresponding second locations on the housing, and,
for a plurality of the functions, the corresponding second location is different from the corresponding first location.

3. A hand-holdable ultrasonic imaging device according to claim 2 wherein:
in each of the first and second modes, the corresponding first locations and corresponding second locations for the plurality of user controls are locations in a row on the housing; and
an order of the corresponding first locations in the row is reversed relative to an order of the corresponding second locations in the row.

4. A hand-holdable ultrasonic imaging device according to claim 1 wherein the housing has a front face, the first and second locations are on the front face and the first and second locations are symmetrical relative to a centerline of the front face extending generally perpendicular to the transducer array.

5. A hand-holdable ultrasonic imaging device according to claim 1 wherein the housing is dimensioned to be cradled in a user's hand, the first location is adjacent a finger of a user's left hand when the housing is cradled in the user's left hand, and the second location is adjacent a corresponding finger of the user's right hand when the housing is cradled in the user's right hand.

6. A hand-holdable ultrasonic imaging device according to claim 1 wherein the display comprises a touch-sensitive display wherein the first and second user controls comprise first and second areas on the touch-sensitive display.

7. A hand-holdable ultrasonic imaging device according to claim 6 wherein the touch-sensitive display covers substantially all of a front face of the device.

8. A hand-holdable ultrasonic imaging device according to claim 1 comprising a mode selection switch operable by a user to switch between the first and second modes.

9. A hand-holdable ultrasonic imaging device according to claim 1 comprising:
a plurality of controls outside of and adjacent to the display;
wherein the control circuits are configured to display labels corresponding to each of the plurality of controls on the display adjacent to the controls;
the control circuits are configured to invoke a function associated with one of the controls when the one of the controls is actuated;
when the device is in the first mode, the first function is associated with one of the controls and a label corresponding to the function is displayed on the display adjacent to the one of the controls; and,
when the device is in the second mode, the first function is associated with a different one of the controls and the label corresponding to the function is displayed on the display adjacent to the different one of the controls.

10. A hand-holdable ultrasonic imaging device according to claim 1 wherein the control circuits are configured to display an image derived from ultrasound data on the display.

11. A hand-holdable ultrasonic imaging device according to claim 10 wherein the control circuits are configured to selectively display the image in a first orientation or a second orientation rotated relative to the first orientation.

12. A hand-holdable ultrasonic imaging device according to claim 11 wherein the display comprises a touch-sensitive display wherein the control circuits are configured to switch between displaying the image in the first orientation and the second orientation in response to a touch on an orientation control defined on an area of the touch-sensitive display.

13. A hand-holdable ultrasonic imaging device according to claim 12 wherein the orientation control is defined on the same area of the touch-sensitive display when the image is displayed in the first orientation as when the image is displayed in the second orientation.

14. A hand-holdable ultrasonic imaging device according to claim 11 wherein the control circuits are configured to determine a direction of motion of the transducer over a subject relative to a longitudinal axis of the transducer and to select between displaying the image in the first orientation and displaying the image in the second orientation based on the direction of motion.

15. A hand-holdable ultrasonic imaging device according to claim 11 comprising an optical sensor configured to sense a direction of motion of the transducer array over a subject wherein the control circuits are configured to select between displaying the image in the first orientation and displaying the image in the second orientation based on the direction of motion sensed by the optical sensor.

16. A hand-holdable ultrasonic imaging device according to claim 10 wherein:
the display comprises a touch-sensitive display,
the image is displayed within a first area on the display,
a second area of the display outside of the first area is configured as a touch pad,
the control circuits are configured to display a cursor on the image and to control a location of the cursor on the image in response to patterns of touch detected in the second area.

17. A hand-holdable ultrasonic imaging device according to claim 16 wherein the control circuits are configured to provide Doppler processing of ultrasound data corresponding to a location on the image at which the cursor is located.

18. A hand-holdable ultrasonic imaging device according to claim 16 wherein the control circuits are configured to:
display an additional cursor on the image;
control a location of the additional cursor on the image in response to patterns of touch detected in the second area; and
compute a distance between the cursor and the additional cursor.

19. A hand-holdable ultrasonic imaging device according to claim 6 operable in a plurality of imaging modes, each of the imaging modes having an associated set of user controls, wherein the control circuits are configured to receive user input selecting one of the plurality of imaging modes and to display on the touch-sensitive display the set of user controls associated with the selected imaging mode.

20. A hand-holdable ultrasonic imaging device according to claim 6 wherein the control circuits are configured to perform functions in response to detecting touch gestures corresponding to the functions on the touch-sensitive display.

21. A hand-holdable ultrasonic imaging device according to claim 6 wherein the control circuits are configured to:
acquire ultrasound data;
process the ultrasound data to generate a sequence of images; and
display the images of the sequence of images in sequence on the display.

22. A hand-holdable ultrasonic imaging device according to claim 21 wherein the control circuits are configured to:
detect motion of a location along a trajectory at which a pressure is applied to the touch-sensitive display;
determine a direction of the motion; and
display the images of the sequence of images in ascending or descending sequence depending upon the direction of the motion.

23. A hand-holdable ultrasonic imaging device according to claim 21 wherein the control circuits are configured to:
determine a speed of the motion; and,
display the images of the sequence of images at a rate based at least in part on the speed of the motion.

24. A device according to claim 1 comprising a data store wherein the device is configured to acquire and store in the data store a sequence of ultrasound images and the device is configured to display the sequence of ultrasound images in sequence on the display as a moving picture or cineloop.

25. A device according to claim 24 wherein the device is configured to control a direction of playback of the moving picture or cineloop in response to a motion of a user's finger on the display.

26. A device according to claim 24 wherein the device is configured to control a rate of playback of the moving picture or cineloop in response to a motion of a user's finger on the display.

27. A method for operating an ultrasonic imaging device comprising a transducer and a display on a hand-holdable unit, the method comprising:
switching the imaging device between a first mode wherein a first function is assigned to a first user control supported on the housing at a first location and a second mode wherein the first function is assigned to a second user control supported on the housing at a second location.

28. A method for operating an ultrasonic imaging device comprising a transducer and a display on a hand-holdable unit, the method comprising:
moving the transducer across a body of a subject while operating the transducer to acquire ultrasound data;
determining a direction of motion of the transducer across the subject relative to an orientation of the transducer;
processing the ultrasound data to generate image data; and
displaying the image data on the display in an orientation based upon the direction of motion of the transducer across the subject.
